(19) United States
(12) Patent Application Publication
(10) Pub. No.: US 2006/0155289 A1
(43) Pub. Date: Jul. 13, 2006

(54) Apparatus for reaming bone cavities

(75) Inventors: Reinhard Windhager, Graz (AT); Ronald Prager, Bovenau (DE); Bernd Robioneck, Preetz (DE)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Stryker Trauma GmbH, Schonkirchen (DE)

(21) Appl. No.: 11/297,060

(22) Filed: Dec. 8, 2005

(30) Foreign Application Priority Data
Dec. 10, 2004 (DE) 20 2004 019 105.9

(51) Int. Cl. A61B 17/16

(52) U.S. Cl. 606/80

(57) ABSTRACT

A device for reaming of a bone cavity or a channel of a hollow bone has an elongated, hollow shaft, a first end of which is adapted to be mounted to a rotary drive means in an angularly stable fashion. The device has a spindle, which is borne rotatably within the shaft and has a first end which extends to the mounting-end of the shaft and a second end which extends beyond the other end of the shaft. The first end of spindle is adapted for a rotary connection to a drive spindle of the rotary device means. A flexible, elongated member is provided which is, at its first end, laterally connectable to the second end of spindle, and a profile member made of a hard material located at the other end of the flexible member.

### BACKGROUND OF THE INVENTION

The invention relates to an apparatus for reaming of bone cavities which has a pliable cutter which cuts bone from the inside out upon rotation of the cutter.

Especially for bones affected by tumors, it is a necessity to remove the cancerous material. To this end, it is known to spoon out the affected bone section. Such a method is laborious and requires access to the treatment location through flesh and tissue.

Further, it is known to ream a hollow bone axially by means of a drill or a reaming device, and to remove the loosened material by suction. Drilling, however, leads to a severe stress of the cortical bone due to pressure developed during reaming.

The present invention is based on solving the problem by creating an apparatus for reaming of bone cavities, for example a channel of a hollow bone, that permits the reaming of the bone efficiently within shortest time and without stressing the bone too much. This is accoOmplished by a flexible cord which extends circumferentially outward upon rotation of a spindle holding the cord.

### SUMMARY OF THE INVENTION

The apparatus of the invention is provided with an elongated, hollow shaft. The shaft can be of a constant diameter along its length, and can have an extension to be clamped in a rotatably fixed manner to a rotary drive tool. The elongated shaft is provided with an axial through-bore, which can have a constant diameter along its length. The shaft can be made of a suitable metal or of plastic.

The shaft serves as a bearing and housing for a spindle which is rotatably mounted in the shaft. Preferably, the spindle is secured against axial movement in the shaft. One end of the spindle extends towards the clamping-end of the shaft, and it is adapted such that it can be connected with a drive spindle of a rotary drive means. The other end of the spindle extends beyond the corresponding end of the shaft. An elongated, flexible member, in particular made of plastic or metal, for example with the shape of a fiber or a rope, is connected laterally to the free end of the spindle. A profile member made of a hard material is connected to the other end of the flexible member.

In use, such an apparatus or such a tool is clamped with the one end of the shaft in a rotary drive tool. This can be designed in a way that its drive spindle automatically gets in rotary engagement with the corresponding end of the spindle. The rotary drive tool at the same time serves as a guide for the tool of the invention. If the rotary drive tool is set in operation, the spindle will rotate, and in this way the profile member will be slung circularly about the axis of the spindle. The profile member, which is preferably made of metal and provided with sharp edges, reams the cancellous bone and loosens the spongy material, so that it can be removed out of the bone by means of a suitable device, for example with a suitable suction device. It shall be understood that a suction device can also be integrated with the tool of the invention.

In order to fix the flexible member, an aspect of the invention envisions that the second end of the spindle is provided with a transverse bore for receiving the corresponding end of the flexible member. A fastening screw is screwed in a threaded hole from the free end of the spindle and clamps the corresponding end of the flexible member in the transverse bore. It shall be understood that flexible members of different length or flexible members clamped to a different length can be used in order to be adapted to the diameter to be reamed in the bone.

The flexible member can consist of plastic or metal. The spindle can be flexible, and a set of spindles of different lengths can be provided for adaptation to different lengths to be reamed.

The above aspects of the invention are provided by an apparatus for reaming the bone cavity including an elongated hollow shaft having a first end adapted to be mounted to a rotary drive tool in an angularly stable fashion. The hollow shaft has a second end which extends into the bone canal or cavity. A spindle is rotatably mounted within the hollow shaft for either rotation with respect thereto or in conjunction therewith depending whether both the hollow shaft and spindle rotate together or only the spindle rotates. The spindle has a first end adjacent the first end of the hollow shaft and a second end which extends into the bone canal with the second end extending beyond the second end of the hollow shaft. A flexible elongate member having a first end laterally connectable to the second of the spindle includes a profile or cutter member made of a hard material mounted on a second end of the flexible member.

In the preferred embodiment, the second end of the flexible member extends through a bore in the spindle generally transverse to the longitudinal axis of the spindle and is held in position by the impingement of a fastening screw which is threaded through an opening in the second end of the spindle. Preferably the fastening screw engages the threads and engages the inner end of the flexible member in the transverse bore.

The flexible member is preferably a fiber or rope made of metal or plastic. The profile member at the outer end of the flexible member is preferably made of metal and has sharp edges. A tip may be provided which includes a series of spindles which are slidably received within the hollow elongate shaft with the spindles being of different lengths. The spindles may be flexible over at least part of their lengths.

### BRIEF DESCRIPTION OF THE DRAWINGS

FIG. 1 shows a side view of an apparatus of the invention; and

FIG. 2 shows the apparatus of FIG. 1 in a position rotated by 90°.

### DETAILED DESCRIPTION

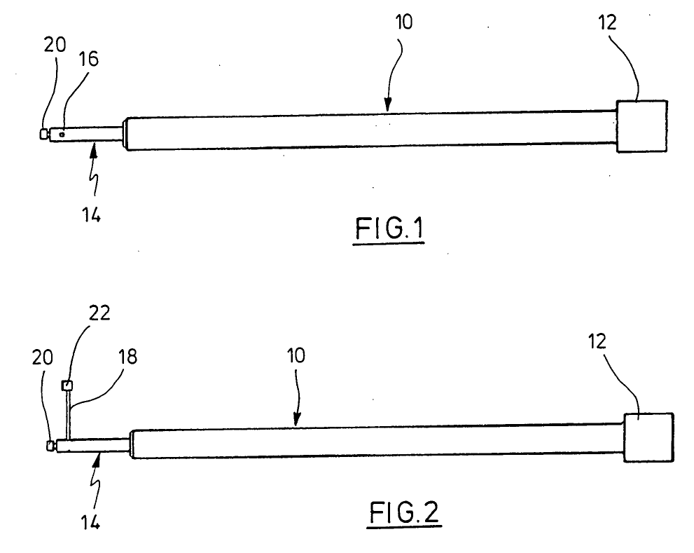

In FIGS. 1 and 2, an elongated shaft 10 can be seen, which is of cylindrical cross-section and exhibits a constant outer diameter over the major part of its length. At the right end as shown in FIGS. 1 and 2, shaft 20 exhibits an extended section 12. This section can be mounted to a rotary driver (not shown) in an angularly stable fashion. Shaft 10 has an axial bore extending end-to-end. This through-bore 15 receives a spindle 14, which extends in FIGS. 1 and 2 with its major part of its length within the shaft 10, namely up to section 12. The right end of the spindle 14 is formed (not shown) such that it can be coupled in a rotary connection to a driven spindle not shown of a rotary drive means. The left end of the spindle 14 extends beyond the left end of shaft 10. In the ending section, the free end of spindle 14 exhibits a transverse bore 16. The bore receives a corresponding end of the flexible member 18. Starting from the free end of the free end section (left end in FIGS. 1 and 2) of spindle 14, a threaded bore is formed in an axial fashion, in which a fastening screw 20 is screwed in. By means of the fastening screw 20, the flexible member is clamped in the transverse bore 16.

If the apparatus shown in FIGS. 1 and 2 is mounted to a rotary drive means and if this is set in operation, the spindle 14 will rotate in shaft 10 and will in this way sling a profile member 22 at the other end of the flexible member 18 in a plane perpendicular to the drawing plane. The profile member 22, which is made of metal and provided with relatively sharp edges, thereby reams a bone cavity or a bone channel, when the apparatus shown is inserted for example into the bone cavity or bone channel which is affected by tumors.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. An apparatus for reaming of a bone cavity or a channel of a hollow bone, comprising:
an elongated, hollow shaft, a first end of which is adapted to be mounted to a rotary drive means in an angularly stable fashion;
a spindle, which is rotatably mounted in the hollow shaft and which extends with a first end to the first end of the shaft and has a second extend extending beyond the second end of the hollow shaft, wherein the first end of spindle is adapted for a rotary connection to a drive spindle of the rotary drive means;
a flexible, elongated member having a first end thereof laterally connectable to the second end of spindle; and
a profile member made of a hard material mounted to a second end of the flexible member.

2. The apparatus according to claim 1, wherein the second end of the spindle has a transverse bore for receiving the first end of the flexible member, and a fastening screw which can be screwed in from the second end of the spindle in an axial threaded bore for clamping the first end of the flexible member in the transverse bore.

3. The apparatus according to claim 1, wherein in that the flexible member is a fiber or a rope.

4. The apparatus according to claim 1 wherein the flexible member is made of plastic or metal.

5. The apparatus according to claim 1 wherein the profile member is made of metal and has sharp edges.

6. The apparatus according to claim 1 wherein a set of spindles of different lengths is provided.

7. The apparatus according to claim 1 wherein the spindle is flexible at least over a part of its length.

8. A reamer for reaming a bore cavity comprising:
a longitudinally extending rotatable spindle having a first end for insertion into a bone cavity and a second end for connection to a rotary drive source; and
a flexible cutter mounted on the first end of said spindle, said cutter extending in a plane generally perpendicular to the longitudinal extend of said spindle rotation thereof.

9. The apparatus according to claim 8, wherein the first end of the spindle has a threaded bore open to the first end and has a transverse bore for receiving the first end of the flexible cutter, the threaded bore receiving a fastening screw that can be screwed in from the first end of the spindle in the threaded bore for clamping the first end of the flexible member in the transverse bore.

10. The apparatus according to claim 8, wherein the flexible cutter is a fiber or a rope.

11. The apparatus according to claim 10 wherein the flexible cutter is made of plastic or metal.

12. The apparatus according to claim 8 wherein a profile member mounted at an outer end of the flexible cutter is made of metal and has sharp edges.

13. The reamer as set forth in claim 8 wherein said flexible cutter has a metal cutting element mounted at an outer end thereof.

14. The apparatus according to claim 8 wherein a set of spindles of different lengths is provided.

15. The apparatus according to claim 8 wherein the spindle is flexible at least over a part of its length.

16. A reamer for reaming bone cavities comprising:
an axially extending hollow shaft having a first end for insertion into bone;
a flexible rotatable spindle for insertion into said hollow shaft having a first end which extends beyond the first end of the hollow shaft; and
a flexible cutter having an inner end mounted adjacent the first end of the rotatable spindle, the cutter having a profile cutting member attached to an outer end thereof.

17. The apparatus according to claim 16, wherein the flexible cutter is a fiber or a rope.

18. The apparatus according to claim 17 wherein the flexible cutter is made of plastic or metal.
